(19) United States
(12) Patent Application Publication
(10) Pub. No.: US 2005/0026847 A1
(43) Pub. Date: Feb. 3, 2005

(54) Methods for treating, preventing, or inhibiting injuries, cell membrane stabilization, and calcium mobilization using pseudopterosin compounds

(76) Inventors: Robert S. Jacobs, Santa Barbara, CA (US); Laura D. Mydlarz, Ithaca, NY (US); Claudia Moya, Santa Barbara, CA (US)

Correspondence Address: Suzannah K. Sundby;Smith, Gambrell & Russell, Suite 800, 1850 M Street, Washington, DC, 20036, US

(21) Appl. No.: 10/899,054

(22) Filed: Jul. 27, 2004

Related U.S. Application Data
(60) Provisional application No. 60/490,267, filed on Jul. 28, 2003.
(60) Provisional application No. 60/491,256, filed on Jul. 31, 2003.
(60) Provisional application No. 60/545,940, filed on Feb. 20, 2004.

(51) Int. Cl. A61K 31/704; A61K 31/70

(52) U.S. Cl. 514/25

(57) ABSTRACT

Disclosed herein are methods for treating, preventing, or inhibiting injuries, methods for inducing, increasing, or modulating calcium mobilization, methods for preventing, inhibiting, decreasing, or modulating phagocytosis, and treating, preventing, or inhibiting diseases and disorders associated with calcium mobilization and phagocytosis. These methods comprise the administration of at least one pseudopterosin compound.

### CROSS REFERENCE TO RELATED APPLICATIONS

This application claims the benefit of U.S. Provisional Patent Application No. 60/490,267, filed 28 Jul. 2003, No. 60/491,256, filed 31 Jul. 2003, and No. 60/545,940, filed 20 Feb. 2004, listing Robert S. Jacobs, Laura Mydlarz, and Claudia Moya as joint inventors, all of which are herein incorporated by reference in their entirety.

### BACKGROUND OF THE INVENTION

1. Field of the Invention

The present invention generally relates to methods for treating, preventing, or inhibiting injuries, cell membrane stabilization, and calcium mobilization using pseudopterosin compounds.

2. Description of the Related Art

Pseudopterosin compounds are a group of diterpene glycosides which were first isolated and characterized from extracts of Pseudopterogorgia elisabethae. Many of the pseudopterosin compounds have been found to exhibit anti-inflammatory, anti-proliferative, and analgesic activities. There are in excess of fifteen such pseudopterosin compounds that have been isolated and characterized in extracts of P. elisabethae as well as in extracts of Symbiodinium spp. See Look, S. A, et al. (1986) J. Organic Chem. 51:5140-5145; Look, S. A, et al. (1986) PNAS 83:6238-6240; Look, S. A, et al. (1986) Tetrahedron 43:3363-3370; Roussis, V., et al. (1990) J. Organic Chem. 55:4922-4925; and United States Patent Application Publication No. 20030104007.

Various pseudopterosin compounds have been known and studied for years. The complete realm of all the biological activities and mechanisms of action of pseudopterosin compounds is yet to be appreciated and understood.

### SUMMARY OF THE INVENTION

The present invention relates to pseudopterosin compounds and methods of using thereof.

In some embodiments, the present invention provides methods for preventing, inhibiting, decreasing, or modulating phagocytosis in a cell which comprises administering to the cell an effective amount of at least one pseudopterosin compound. In some embodiments, the cell may be a Tetrahymena spp. cell or a Heterocapsa spp. cell. The pseudopterosin compound maybe Pseudopterosin A (PsA), Pseudopterosin B (PsB), Pseudopterosin C (PsC), Pseudopterosin D (PsD), Pseudopterosin E (PsE), Pseudopterosin F (PsF), Pseudopterosin G (PsG), Pseudopterosin H (PsH), Pseudopterosin I (PsI), Pseudopterosin J (PsJ), Pseudopterosin K (PsK), Pseudopterosin L (PsL), Pseudopterosin M (PsM), Pseudopterosin N (PsN), Seco-Pseudopterosin A (SPsA), Seco-Pseudopterosin B (SPsB), Seco-Pseudopterosin C(SPsC), Seco-Pseudopterosin D (SPsD), Seco-Pseudopterosin E (SPsE), or Elisabethatriene, preferably the pseudopterosin compound is Pseudopterosin A (PsA), Pseudopterosin B (PsB), Pseudopterosin C (PsC), or Pseudopterosin D (PsD), and more preferably the pseudopterosin compound is Pseudopterosin A (PsA). In some embodiments, the effective amount ranges from about 0.1 μM to about 100 μM, preferably about 1 μM to about 50 μM, more preferably about 2 μM to about 25 μM, and even more preferably about 2.5 μM to about 10 μM. In some embodiments, the present invention further comprises administering a calcium ionophore, an inhibitor of PLC activation, or both.

In some embodiments, the present invention provides methods of treating, preventing, or inhibiting a disease or disorder associated with phagocytosis in a subject which comprises administering to the subject a therapeutically effective amount of at least one pseudopterosin compound.

In some embodiments, the present invention provides methods for inducing, increasing, or modulating calcium mobilization in a cell which comprises administering to the cell an effective amount of at least one pseudopterosin compound. In some embodiments, the cell maybe a Tetrahymena spp. cell or a Heterocapsa spp. cell. In some embodiments, the pseudopterosin compound is Pseudopterosin A (PsA), Pseudopterosin B (PsB), Pseudopterosin C (PsC), Pseudopterosin D (PsD), Pseudopterosin E (PsE), Pseudopterosin F (PsF), Pseudopterosin G (PsG), Pseudopterosin H (PsH), Pseudopterosin I (PsI), Pseudopterosin J (PsJ), Pseudopterosin K (PsK), Pseudopterosin L (PsL), Pseudopterosin M (PsM), Pseudopterosin N (PsN), Seco-Pseudopterosin A (SPsA), Seco-Pseudopterosin B (SPsB), Seco-Pseudopterosin C (SPsC), Seco-Pseudopterosin D (SPsD), Seco-Pseudopterosin E (SPsE), or Elisabethatriene, preferably the pseudopterosin compound is Pseudopterosin A (PsA), Pseudopterosin B (PsB), Pseudopterosin C (PsC), or Pseudopterosin D (PsD), and more preferably the pseudopterosin compound is Pseudopterosin A (PsA). In some embodiments, the effective amount ranges from about 0.1 μM to about 100 μM, preferably about 1 μM to about 50 μM, more preferably about 1 μM to about 25 μM, and even more preferably about 1 μM to about 10 μM. In some embodiments, the present invention further comprises administering an inhibitor of PLC activation.

In some embodiments, the present invention provides methods of treating, preventing, or inhibiting a disease or disorder associated with calcium mobilization in a subject which comprises administering to the subject a therapeutically effective amount of at least one pseudopterosin compound.

In some embodiments, the present invention provides methods of treating, preventing, or inhibiting an injury to a cell or a tissue which comprises administering to the subject a therapeutically effective amount of at least one pseudopterosin compound to the cell or the tissue. The injury is a physical injury, a chemical injury, a radiation injury, or a combination thereof. In some embodiments, the pseudopterosin compound is Pseudopterosin A (PsA), Pseudopterosin B (PsB), Pseudopterosin C (PsC), Pseudopterosin D (PsD), Pseudopterosin E (PsE), Pseudopterosin F (PsF), Pseudopterosin G (PsG), Pseudopterosin H (PsH), Pseudopterosin I (PsI), Pseudopterosin J (PsJ), Pseudopterosin K (PsK), Pseudopterosin L (PsL), Pseudopterosin M (PsM), Pseudopterosin N (PsN), Seco-Pseudopterosin A (SPsA), Seco-Pseudopterosin B (SPsB), Seco-Pseudopterosin C(SPsC), Seco-Pseudopterosin D (SPsD), Seco-Pseudopterosin E (SPsE), or Elisabethatriene, preferably the pseudopterosin compound is Pseudopterosin A (PsA), Pseudopterosin B (PsB), Pseudopterosin C (PsC), or Pseudopterosin D (PsD), and more preferably the pseudopterosin compound is Pseudopterosin A (PsA). In some embodiments, the therapeutically effective amount ranges from about 0.1 μM to about 100 μM, preferably about 1 μM to about 50 μM, more preferably about 2.5 μM to about 25 μM, and even more preferably about 5 μM to about 15 μM. In some embodiments, the present invention further comprises administering at least one supplementary active compound.

It is to be understood that both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention and together with the description serve to explain the principles of the invention.

### DESCRIPTION OF THE DRAWINGS

This invention is further understood by reference to the drawings wherein:

FIG. 1 shows T. thermophila with food vacuoles filled with India ink (light microscopy X400).

FIG. 2 is a graph showing the effect of PsA on Tetrahymena phagocytosis.

FIG. 3 is a graph showing the effect of A23187 on Tetrahymena phagocytosis.

FIG. 4 is a graph showing the effect of CaCl₂ on Tetrahymena phagocytosis.

FIG. 5 shows the effect of Pertussis toxin pretreatment on PsA phagocytic activity.

FIG. 6 shows the effect of Pertussis toxin pretreatment on U73122 phagocytic activity.

FIG. 7A shows Tetrahymena cells stained with Calcium Orange under fluorescence microscopy (X400).

FIG. 7B shows Tetrahymena cells treated with PsA and stained with Calcium Orange under fluorescence microscopy (X400).

FIG. 7A shows Tetrahymena cells pretreated with Pertussis toxin, then treated with PsA, and stained with Calcium Orange under fluorescence microscopy (X400).

FIG. 8 shows the effect of Pertussis toxin on PsA activity.

FIG. 9 shows the effect of Pertussis toxin on U73122 activity.

FIG. 10A shows the effect of pertussis toxin pretreatment on Mastoparan activity.

FIG. 10B shows the effect of Suranim pretreatment on PsA activity.

FIG. 11A1 shows the response of Symbiodinium and H. pygmaea to ultrasound induced injury. (A) Epifluorescent micrograph of control Symbiodinium sp. from PE. The micrograph is a red fluorescence indicating the presence of chlorophyll. (B) Epifluorescent micrograph of physically injured Symbiodinium sp. from PE. Control. The micrograph is a green fluorescence indicating the presence of ROS which reacts with DCFH-DA.

FIG. 11A2 shows the response of Symbiodinium and H. pygmaea to ultrasound induced injury. (A) Epifluorescent micrograph of control H. pygmaea. The micrograph is a red fluorescence indicating the presence of chlorophyll. (B) Epifluorescent micrograph of physically injured H. pygmaea. The micrograph is a green fluorescence indicating the presence of ROS which reacts with DCFH-DA. Excitation 488 nm, emmission 510 (longpath).

FIG. 11B1 shows the kinetic he oxidative burst caused by sonic sound in PE Symbiodinium. (n=5) Arrows indicate point of injury.

FIG. 11B2 shows the kinetic he oxidative burst caused by sonic sound in H. pygmaea. (n=5) Arrows indicate point of injury.

FIG. 12 shows the HPLC chromatogram of PsA, PsB, PsC and PsD used in these experiments.

FIG. 13A shows a log-dose response curve for the inhibition of ROS release by pseudopterosins in Heterocapsa pygmaea cells.

FIG. 13B shows the decrease in H₂O₂ production in Heterocapsa pygmaea cells with increased concentration of pseudopterosins, indicating a pseudo-first order kinetic relationship.

FIG. 14 shows that 10 μM of mastoparan had no effect on Symbiodinium spp. cells but did cause a large oxidative burst in H. pygmaea cells.

FIG. 15 shows the reductions in hydrogen peroxide levels by pseudopterosin compounds were not due to direct antioxidant effect.

FIG. 16 shows the inhibition of ROS release by DPI.

FIG. 17 shows the effects of Pertussis Toxin (PT) on the oxidative burst of H. pygmaea caused by physical injury.

FIG. 18 shows the effect of a pseudopterosin mixture (Ps) pretreatment (1 hour) on T. thermophila cells exposed to physical injury (n=3).

### DETAILED DESCRIPTION OF THE INVENTION

Tetrahymena spp. share similar physiological, biochemical, and pharmacological similarities to mammalian macrophages, neutrophils, and mast cells. Therefore, a unicellular ciliate, Tetrahymena thermophila, was used as an experimental model in order to further study the mechanisms of action of pseudopterosin compounds, such as Pseudopterosin A (PsA), and to investigate the signal transduction mechanism involved in phagocytosis as the subcellular regulation of phagosome formation in Tetrahymena spp. is not fully understood.

As disclosed in Examples 1 and 2, U73122 (an inhibitor of PLC activation) and the marine natural product PsA increase calcium release from intracellular stores and decrease the incidence of newly formed phagosomes in T. thermophila. None of the observed effects of U73122 on T. thermophila were inhibited by Pertussis toxin (PT) treatment. In contrast, the effects of PsA on intracellular calcium release and phagosome formation were inhibited following pretreatment with PT. In addition, the effects of PsA were not inhibited by Verapamil or Gd³⁺. Thus, pseudopterosin compounds such as PsA appear to act by a submolecular mechanism at a site coupled to the Gi/o protein that is distinct from the U73122 site of action. Therefore, the present invention provides methods for increasing, inducing, or modulating the release of calcium from intracellular stores in a cell and methods for preventing, inhibiting, decreasing, or modulating the formation of phagosomes in a cell which comprise administering an effective amount of at least one pseudopterosin compound to the cell.

Recently, it has been discovered that Symbiodinium spp. symbionts are involved in the synthesis of pseudopterosin compounds and can produce pseudopterosin compounds without the aid of the host, P. elisabethae. See United States Patent Application Publication No. 20030104007, which is herein incorporated by reference. Symbiodinium spp. cells isolated from P. elisabethae are known to be resistant to rupture and injury. Previous studies have shown that high force levels using a French press at 1200 psi was necessary in order to uniformly rupture the cell membranes of Symbiodinium spp. cell. Thus, as disclosed in Example 3, Example 4, and Example 5, experiments were conducted to determine whether pseudopterosin compounds are responsible for Symbiodinium spp. cells being resistant to injury. As provided herein, Symbiodinium spp. cells having pseudopterosin compounds and its free living related species, H. pygmaea incubated with pseudopterosin compounds were found to be less susceptible to physical and chemical injuries as well as those due to radiation. Therefore, the present invention provides methods for treating, preventing, or inhibiting an injury to a cell which comprises administering an effective amount of at least one pseudopterosin compound to the cell.

As used herein, “pseudopterosin compounds” include natural, synthetic, modified, and substituted pseudopterosins, seco-pseudopterosins, diterpene aglycones, and tricyclic diterpenes that may be produced by, synthesized in, or isolated from species belonging to the genus Pseudopterogorgia, Symbiodinum spp. symbionts, or derivatives thereof such as Pseudopterosin A (PsA), Pseudopterosin B (PsB), Pseudopterosin C (PsC), Pseudopterosin D (PsD), Pseudopterosin E (PsE), Pseudopterosin F (PsF), Pseudopterosin G (PsG), Pseudopterosin H (PsH), Pseudopterosin I (PsI), Pseudopterosin J (PsJ), Pseudopterosin K (PsK), Pseudopterosin L (PsL), Pseudopterosin M (PsM), Pseudopterosin N (PsN), Seco-Pseudopterosin A (SPsA), Seco-Pseudopterosin B (SPsB), Seco-Pseudopterosin C(SPsC), Seco-Pseudopterosin D (SPsD), Seco-Pseudopterosin E (SPsE), and Elisabethatriene. As used herein, “pseudopterosin compositions” include cellular extracts of Symbiodinum spp. symbionts or hosts having pseudopterosin compounds.

Derivatives of pseudopterosin compounds include compounds that have chemical structures and activities that are similar to those compounds produced by, synthesized in, or isolated from Symbiodinum spp. symbionts or hosts thereof. Derivatives of pseudopterosin compounds may be synthesized by derivatizing the various naturally occurring pseudopterosins and seco-pseudopterosins which are isolated from Symbiodinum hosts, such as sea whips, according to known procedures such as those described by Look et al. (1986) PNAS 83:6238-6240; Look et al. (1986) J. Org. Chem. 51:5140-5145; Look et al. (1987) Tetrahedron 43:3363-3370; Roussis et al. (1990) J. Org. Chem. 55:4916-4922; and U.S. Pat. Nos. 4,849,410, 4,745,104, and 5,624,911, which are herein incorporated by reference.

Modified or substituted pseudopterosin compounds include compounds having one group substituted for another group such as a halogen in place of a hydrogen that may alter pseudopterosin potency, stability, activity, and the like. Such modifications or substitutions are known in the art and include other glycoside substitutions such as those found in the biosynthetically related steroid glycosides, Digitalis and Digoxin, and those known in the art. Modifications also include substitutions of sugars of varying chain length, as known in the art, which can alter the pharmacokinetics of the aglycone and thus the suitability of the molecule for various routes of administration as well as the increasing the half-life of the molecule in vivo and its selectivity (bioavailability) for various tissues and organs. Modifications also include those that alter the polarity of the pseudopterosin compounds as the polarity of a compound affects its half-life, thereby affecting its absorption in the kidneys, as known in the art.

One of ordinary skill in the art should be readily able to obtain a variety of pseudopterosin compounds from Symbiodinium spp. symbionts of other hosts by methods known in the art without undue experimentation. For example, pseudopterosin compounds may be obtained from Symbiodinium spp. isolated from hosts such as Aiptasia, Anthopleura, Bartholomea, Cassiopeia, Condylactis, Corbulifera, Corculum, Dichotomia, Discosoma, Gorgonia, Heliopora, Hippopus, Lebrunia, Linuche, Mastigias, Meandrina, Montastraea, Montipora, Oculina, Plexaura, Pocillopora, Pseudoterogorgia, Rhodactis, Stylophora, Tridacna, Zoanthus, and the like. Examples of specific Symbiodinium spp. symbionts include S. kawagutii, S. goreaui, S. muscatinei, S. pulchrorum, S. bermudense, S. californium, S. microadriatiucum, S. pilosum, S. meandrinae, S. corculorum, S. linucheae, and the like. Preferred Symbiodinium spp. belong to phylotype B1 as classified by LaJeunesse, J. Phycol. (2001) 37:866-880, which is herein incorporated by reference.

Additionally, various pseudopterosin compounds may be obtained from symbionts isolated from P. elisabethae found in different geographical locations as different P. elisabethae populations in the Bahamas produce different pseudopterosin compounds. For example, PsA through PsD, were originally found in P. elisabethae populations off Crooked Island in the Bahamas. See Clardy, J. et al. (1986) J. Org. Chem. 51:5140-5145, which is herein incorporated by reference. PsE through PsJ were found in P. elisabethae populations in Bermuda and PsK through PsL were found in populations off Great Abaco Island. See Fenical, W. et al. (1990) J. Org. Chem. 55(16):4916, which is herein incorporated by reference.

The pseudopterosin compounds may be obtained from freshly isolated symbionts. Alternatively, the pseudopterosin compounds may be obtained from cultured or cultivated symbionts such as those from established cultures and cell lines. Cell cultures and cell lines may be made by conventional methods known in the art. See, e.g. LaJeunesse (2001) and Trench, R. K. et al. (2000) J. Exp. Mar. Biol. Ecol. 249:219-233, which are herein incorporated by reference.

The pseudopterosin compounds of the present invention may be for treating, preventing or inhibiting diseases or disorders associated with calcium mobilization.

The pseudopterosin compounds of the present invention may be for treating, preventing or inhibiting diseases or disorders associated with phagocytosis. As used herein, “diseases and disorders associated with phagocytosis” include those relating to bone marrow derived cells that perform phagocytosis such as macrophages, neutrophils, eosinophils, and leukocytes, and those that produce a number of reactive oxygen species in response to various stimuli. See Davey, A. K., et al. (1995) Proceedings—Beltwide Cotton Conferences 1:286-293; Pick, E., et al. (1981) Heterog. Mononucl. Phagocytes, [Proc. Int. Workshop] Meeting Date 1980, 331-338; Bacurau, R. F. P., et al. (1999) Cell Biochem. and Function 17(3):175-182; Baldridge, C. W. and Gerard R. W. (1933) Am. J. Physiol. 103:235-236; Dwyer, S. C., et al. (1996) Biochim. Biophys. Acta. 1289:231-237; Henderson, L. M., et al. (1989) Biochem. J. 264:249-255; and Morel, F., (1991) Eur. J. Biochem. 201:523-546, which are herein incorporated by reference.

Such stimuli include bacterial infection, parasites, venoms of snake, cobra, scorpion, bee, wasp, spider, and the like that may introduce a foreign protein or peptide that would cause over-expression of chemotactic and phagocytic activity and the subsequent inflammation response. See Davey, A. K., et al. (1995) Proceedings—Beltwide Cotton Conferences 1:286-293; Bertholet, S., et al. (2003) Infect. and Immun. 71(4):2095-2101; Handman, E., et al. (2002) Trends in Parasit. 18(8): 332-334; Alam, M. I. and Gomes, A. (1998) Toxicon 36(1):207-215; Fearn, H. J., et al. (1964) J. Pharm. and Pharmacol. 12(2):79-84; Rivers, D. B., et al. (2002) Toxicon 40(1):9-21; Fukuhara, Y. D. M., et al. (2002) Toxicon 41(1): 49-55; Scharf, S. M. (2002) Critical Care Medicine 30(7):1669-1670; Voronov, E., et al. (1999) J of Venomous Animals and Toxins 5(1):5-33; Rees, R. S., et al. (1984) J. Invest. Dermatol. 83(4); Domingos, M. O., et al. (2003) Toxicon 42(5):471-479, which are herein incorporated by reference.

The above mentioned inflammatory response can be initiated in the scalp and all topical sites, membranes of the eye, oral and nasal cavities, lungs, gastro intestinal tract, joints, heart, and circulatory system. The inflammatory response include those stimulated by burns, intestinal parasite infections associated with an inflammatory response, septic shock, and physical wounds from a variety of sources such as abrasions, sun burn, poison oak and poison ivy. See Sayeed, N. M. (1998) Medicina (Buenos Aires) 58(4):386-392; Ehrlich, H. P. (1984) J of Trauma 24(4):311-318; Rosengren, S. and Firestein, G. S. (1997) Purinergic Approaches in Experimental Therapeutics 301-313; Barton, B. E. (1995) Expert Opinion on Therapeutic Patents 5(1):13-21; Matsumura Y. and Ananthaswamy, H. N. (2002) Expert Reviews in Molecular Medicine [electronic resource] 4:1-22; Lorentz, A., et al. (1999) Eur. J. of Immunol. 29(5):1496-1503; Befus, D. and Bienenstock, J. (1984) Contemporary Topics in Immunobiology 12(Immunobiol. Parasites Parasit. Infect.):71-108; Waller, C. W. and Waters, I. W. (1974) U.S. NTIS, PB Rep. (No. 239665) 167 pp.; Guin J. D. (2001) Skin Therapy Letter 6(7):3-5; Kepel, E., et al. (1974) J of Invest. Dermatol. 62(6):595-596; and Sherertz, E. F. (1997) J. Am. Acad. of Dermatol. 36(4):647-649, which are herein incorporated by reference.

Diseases and disorders related to inflammation are known in the art and include psoriases, dermatitis, delayed sensitivity (poison ivy, poison oak, rashes) gout, arthritis, anaphylactic shock, asthma, gastritis, colitis, thrombophlebitis, precancerous polyps of the colon, heart disease, Alzheimer's Disease, and the like.

The pseudopterosin compounds of the present invention may be for treating, preventing or inhibiting an injury. In preferred embodiments, the injury is a cellular or tissue injury. In preferred embodiments, the injury is a chemical injury, a physical injury, a radiation injury, or a combination thereof.

The pseudopterosin compounds of the present invention may be used in combination with or as a substitution for treatments of the above conditions. For example, the compounds of the invention may be used alone or in combination with supplementary active compounds used to treat, prevent, or inhibit injuries such as alpha lipoic acids, reactive oxygen species scavengers such as coenzyme Q, vitamin E, vitamin C, pyruvate, melatonin, niacinamide, N-acetylcysteine, GSH, nitrones, inhibitors of reactive oxygen species, anti-inflammatory agents, antibiotics, antiproliferative agents, analgesics, and the like.

Antiinflammatory agents include aspirin, ibuprofen, acetaminophen, indomethacin, phenylbutazone, gold compounds, steroids, NSAIDS, penicillamine, and the like.

Antibiotics include penicillin, cloxacillin, dicloxacillin, methicillin, nafcillin, oxacillin, ampicillin, amoxicillin, bacampicillin, azlocillin, carbenicillin, mezlocillin, piperacillin, ticarcillin, azithromycin, clarithromycin, clindamycin, erythromycin, lincomycin, demeclocycline, doxycycline, minocycline, oxytetracycline, tetracycline, quinolone, cinoxacin, nalidixic acid, fluoroquinolone, ciprofloxacin, enoxacin, grepafloxacin, levofloxacin, lomefloxacin, norfloxacin, ofloxacin, sparfloxacin, trovafloxacin, bacitracin, colistin, polymyxin B, sulfonamide, trimethoprim-sulfamethoxazole, co-amoxyclav, cephalothin, cefuroxime, ceftriaxone, vancomycin, gentamicin, amikacin, metronidazole, chloramphenicol, nitrofurantoin, co-trimoxazole, rifampicin, isoniazid, pyrazinamide, and the like.

Antiproliferative agents include altretamine, amifostine, anastrozole, arsenic trioxide, bexarotene, bleomycin, busulfan, capecitabine, carboplatin, carmustine, celecoxib, chlorambucil, cisplatin, cisplatin-epinephrine gel, cladribine, cytarabine liposomal, daunorubicin liposomal, daunorubicin daunomycin, dexrazoxane, docetaxel, doxorubicin, doxorubicin liposomal, epirubicin, estramustine, etoposide phosphate, etoposide VP-16, exemestane, fludarabine, fluorouracil 5-FU, fulvestrant, gemicitabine, gemtuzumab-ozogamicin, goserelin acetate, hydroxyurea, idarubicin, ifosfamide, imatinib mesylate, irinotecan, letrozole, leucovorin, levamisole, liposomal daunorubicin, melphalan L-PAM, mesna, methotrexate, methoxsalen, mitomycin C, mitoxantrone, paclitaxel, pamidronate, pegademase, pentostain, porfimer sodium, streptozocin, talc, tamoxifen, temozolamide, teniposide VM-26, topotecan, toremifene, tretinoin, ATRA, valrubicin, vinorelbine, zoledronate, and the like.

Analgesics include opioids such as morphine, codeine, semi-synthetics including meperidine (Demerol), propoxyphen (Darvon), and the like, NSAIDS, acetaminophen, aspirin, ibuprofen, diclofenac, ketoprofen, and the like.

A compound of the present invention may be administered in a therapeutically effective amount to a mammal such as a human. A therapeutically effective amount may be readily determined by standard methods known in the art. As defined herein, a therapeutically effective amount of a compound of the invention ranges from about 0.1 to about 25.0 mg/kg body weight, preferably about 1.0 to about 20.0 mg/kg body weight, and more preferably about 10.0 to about 20.0 mg/kg body weight. Preferred topical concentrations include about 0.1% to about 20.0% in a formulated salve. As used herein, an “effective amount” refers to an amount that provides an observable desired change as compared with a control. For example, if the desired change is a decrease the amount of a given protein and administration of 0.9 μM of a compound does not produce an observable decrease as compared with a control, but the administration of 1 μM does produce an observable decrease, then the effective amount is about 1 μM or more.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of the compound can include a single treatment or, preferably, can include a series of treatments.

In a preferred example, a subject is treated with a compound of the invention in the range of between about 0.1 to about 25.0 mg/kg body weight, at least one time per week for between about 5 to about 8 weeks, and preferably between about 1 to about 2 weeks. It will also be appreciated that the effective dosage of the compound used for treatment may increase or decrease over the course of a particular treatment. Changes in dosage may result and become apparent by standard diagnostic assays known in the art. In some conditions chronic administration may be required.

The pharmaceutical compositions of the invention may be prepared in a unit-dosage form appropriate for the desired mode of administration. The compositions of the present invention may be administered for therapy by any suitable route including oral, rectal, nasal, topical (including buccal and sublingual), vaginal and parenteral (including subcutaneous, intramuscular, intravenous and intradermal). It will be appreciated that the preferred route will vary with the condition and age of the recipient, the nature of the condition to be treated, and the chosen active compound.

It will be appreciated that the actual dosages of the agents used in the compositions of this invention will vary according to the particular complex being used, the particular composition formulated, the mode of administration, and the particular site, host, and disease being treated. Optimal dosages for a given set of conditions may be ascertained by those skilled in the art using conventional dosage-determination tests in view of the experimental data for a given compound. Administration of prodrugs may be dosed at weight levels that are chemically equivalent to the weight levels of the fully active forms.

The pseudopterosin compounds of the present invention can be incorporated into pharmaceutical compositions suitable for administration. Pharmaceutical compositions of this invention comprise an therapeutically effective amount of at least one pseudopterosin compound of the present invention and an inert, pharmaceutically acceptable carrier or diluent. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Supplementary active compounds include other pseudopterosins and seco-pseudopterosins such as those described in U.S. Pat. Nos. 4,745,104, 4,849,410, and 5,624,911, all of which are herein incorporated by reference. Supplementary compounds also include hydrocortisone, cox inhibitors such as indomethacin or salicylates, fixed anesthetics such as lidocaine, opiates, and morphine.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent is dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, at least one pseudopterosin compound is dissolved in DMSO and diluted with water.

The composition may also be in the form of a solution of a salt form of the active ingredient in an appropriate aqueous vehicle such as water or isotonic saline or dextrose solution.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration intranasally or by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical compositions suitable for injectable use include sterile aqueous solutions (where water soluble) or dispersions and sterile powders for the extemporaneous preparation of sterile injectable solutions or dispersion. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a therapeutically effective amount of a compound of the invention in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active compound plus any additional desired ingredient from a previously sterile-filtered solution thereof.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, foams, powders, sprays, aerosols or creams as generally known in the art.

For example, for topical formulations, pharmaceutically acceptable excipients may comprise solvents, emollients, humectants, preservatives, emulsifiers, and pH agents. Suitable solvents include ethanol, acetone, glycols, polyurethanes, and others known in the art. Suitable emollients include petrolatum, mineral oil, propylene glycol dicaprylate, lower fatty acid esters, lower alkyl ethers of propylene glycol, cetyl alcohol, cetostearyl alcohol, stearyl alcohol, stearic acide, was, and others known in the art. Suitable humectants include glycerin, sorbitol, and others known in the art. Suitable emulsifiers include glyceryl monostearate, glyceryl monoleate, stearic acid, polyoxyethylene cetyl ether, polyoxyethylene cetostearyl ether, polyoxyethylene stearyl ether, polyethylene glycol stearate, and others known in the art. Suitable pH agents include hydrochloric acid, phosphoric acid, diethanolamine, triethanolamine, sodium hydroxide, monobasic sodium phosphate, dibasic sodium phosphate, and others known in the art. Suitable preservatives include benzyl alcohol, sodium benzoate, parabens, and others known in the art.

For administration to the eye, the compound of the invention is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/cilary, lens, choroid/retina and selera. The pharmaceutically acceptable ophthalmic vehicle may be an ointment, vegetable oil, or an encapsulating material. A compound of the invention may also be injected directly into the vitreous and aqueous humor.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the pseudopterosin compounds of the present invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

In some embodiments, the pseudopterosin compounds of the present invention may be prepared with carriers that will protect the compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The pseudopterosin compounds and pseudopterosin compositions of the present invention may be provided in kits along with instructions for use. The kits may further include supplementary active compounds, wound dressings, applicators for administration, or combinations thereof.

The pseudopterosin compounds of the present invention may be prepared using the reaction routes and synthesis schemes known in the art, employing the techniques available in the art using starting materials that are readily available. For example, a variety of pseudopterosin compounds may be made by obtaining elisabethatriene from cultures of at least one Symbiodinium spp. symbiont and then chemically modifying elisabethatriene by conventional methods in the art. See e.g., Look et al. (1986) PNAS 83:6238-6240; Look et al. (1986) J. Org. Chem. 51:5140-5145; Look et al. (1987) Tetrahedron 43:3363-3370; Roussis et al. (1990) J. Org. Chem. 55:4916-4922; and U.S. Pat. Nos. 4,849,410, 4,745,104, and 5,624,911. Occasionally, the reaction routes and synthesis schemes known in the art may not be applicable to each compound included within the disclosed scope of the invention. The compounds for which this occurs will be readily recognized by those skilled in the art. In all such cases, either the reactions can be successfully performed by conventional modifications.

The following examples are intended to illustrate but not to limit the invention.

### EXAMPLE 1

### Phagocytosis Activity Assay

Log-phase T. thermophila cultures were grown in 2% protease peptone, at 25° C. The cells were washed and resuspended in 10 mM HEPES, pH 7.4 (250,000 cells/ml). Test compounds were mixed with diluted India ink (1:25, v:v) in order to visualize the newly formed phagosomes. At t=0, the drug/ink mixture was added to the Tetrahymena cells, and at t=10 minutes, 500 ml samples of cell suspension were removed and fixed in 10% formaldehyde. At least about 100 random cells from each sample were then examined using a light microscope. Phagocytic activity was assessed by calculating the ratio of cells with food vacuoles compared to the cells with no food vacuoles. FIG. 1 shows T. thermophila with food vacuoles filled with India ink (light microscopy X400).

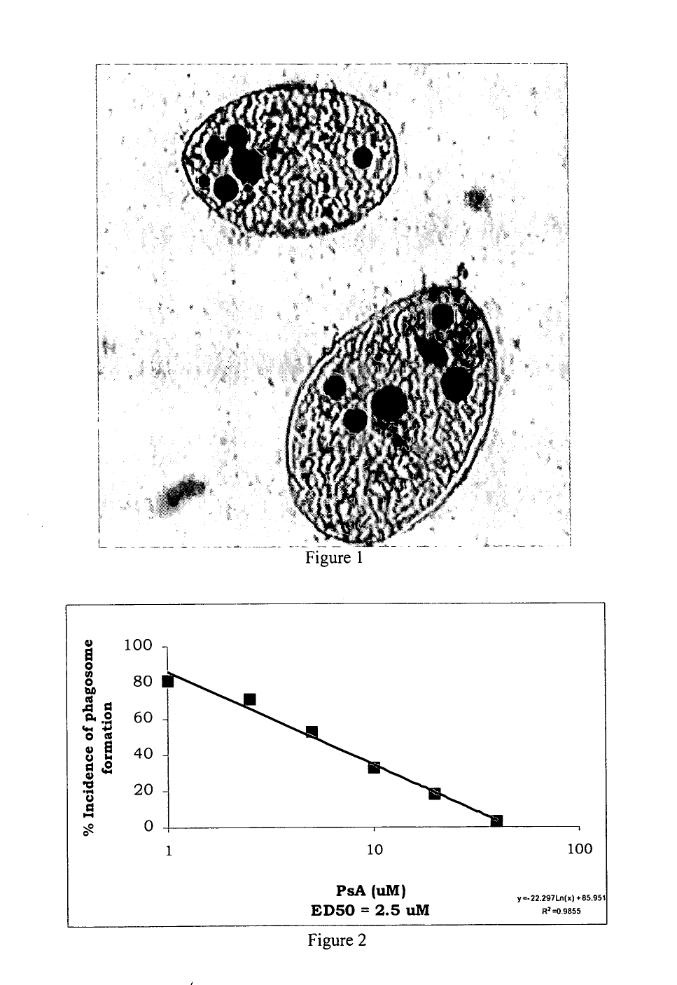

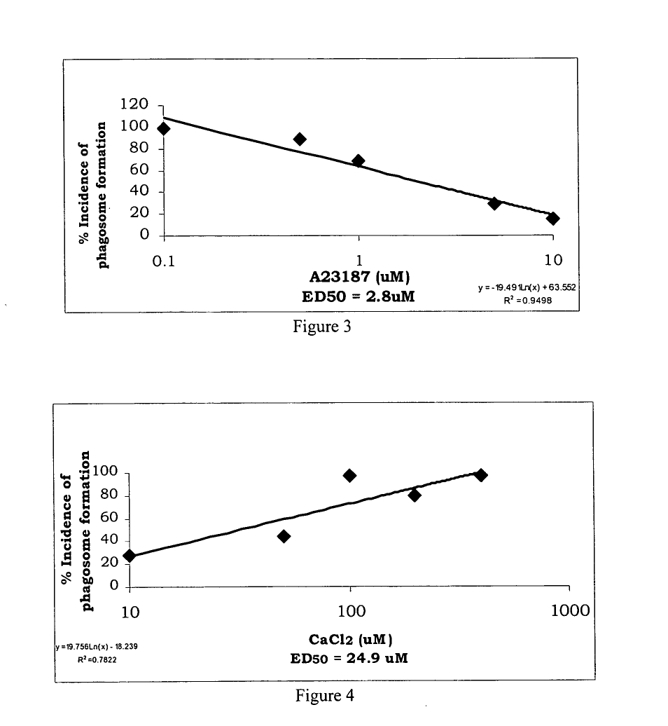

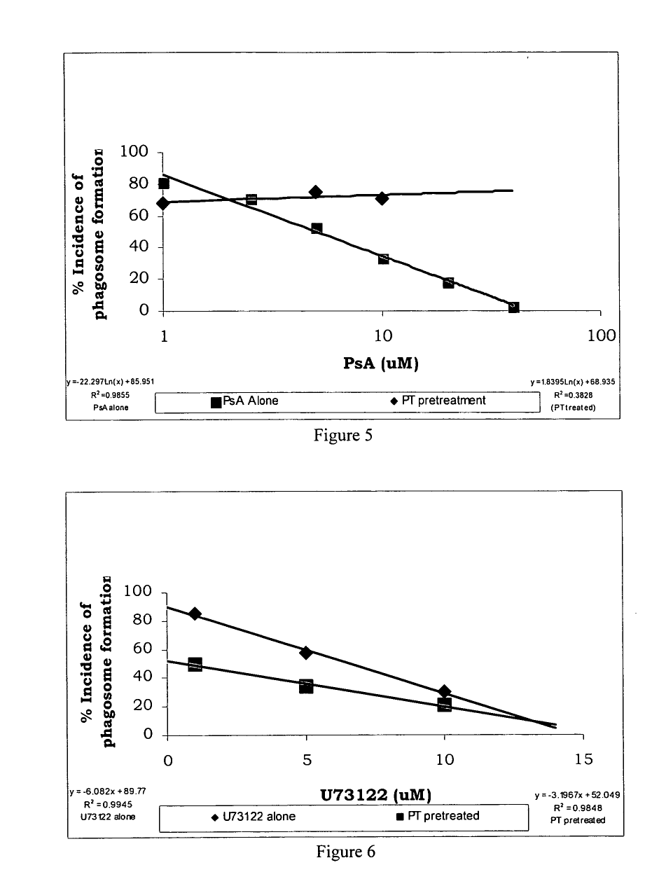

FIG. 2 shows that PsA decreases the rate of phagosome formation in a dose dependent manner. FIG. 3 shows that the calcium inonophore, A23187, decreases the rate of phagosome formation in a dose dependent manner. FIG. 4 shows that CaCl₂ increases the rate of phagocytic activity in Tetrahymena cells. FIG. 5 shows that Pertussis toxin pretreatment (5 minutes) completely blocks the effect of PsA on phagocytosis. FIG. 6 shows that U73122 decreases the rate of phagosome formation in a dose dependent manner (ED₅₀=4.3 μM), but it is not blocked by Pertussis toxin pretreatment.

### EXAMPLE 2

### Ca⁺⁺ Signaling Assay

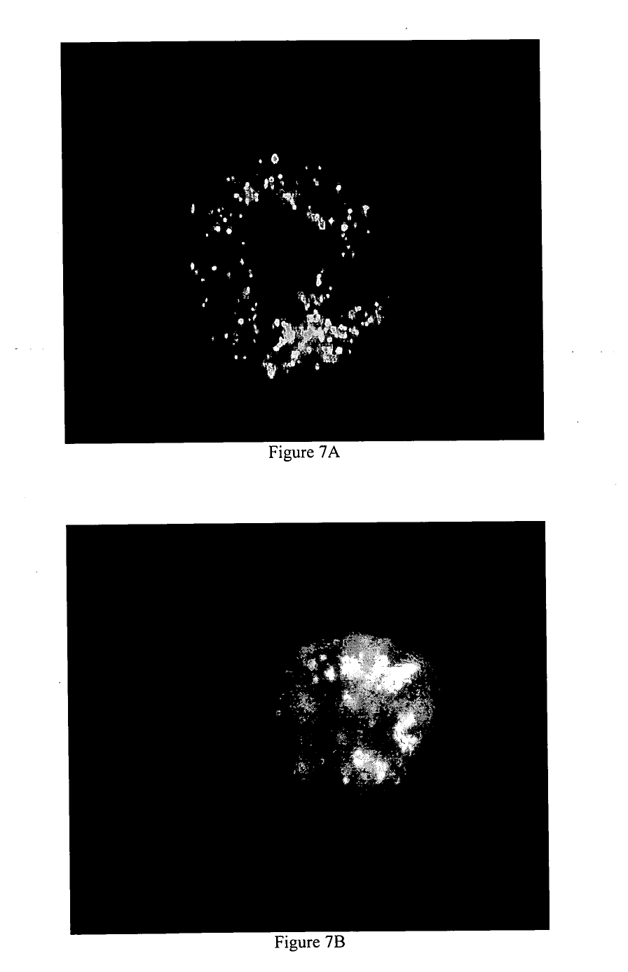

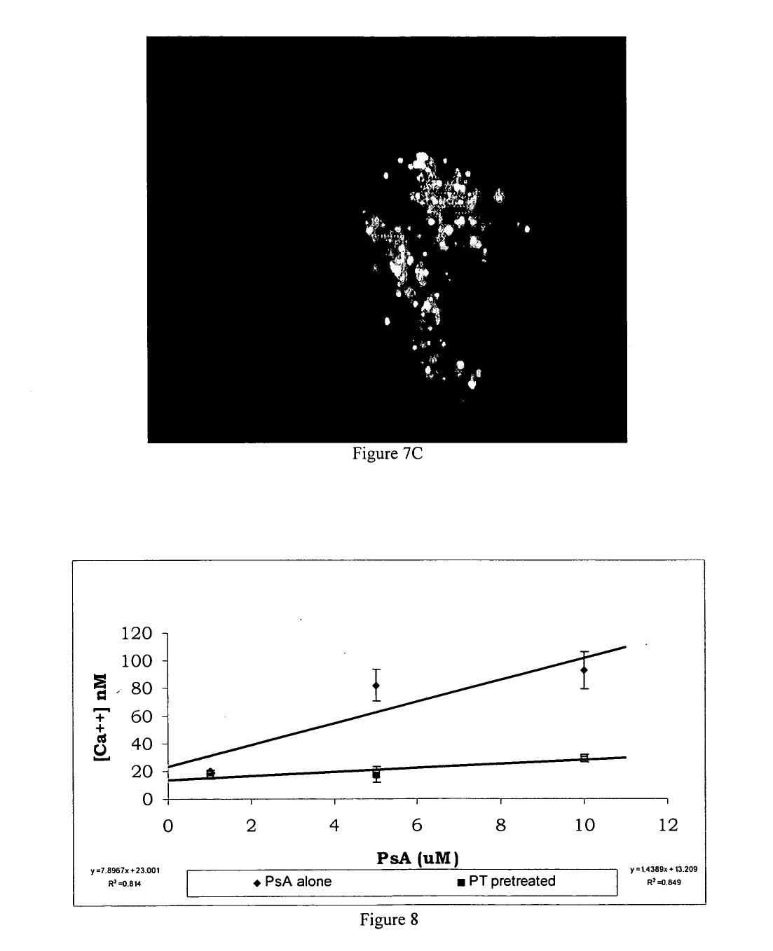

Cell density was adjusted to 500,000 cells/ml using a 10 mM HEPES and 2.5 mM Probenecid buffer (pH 7.4) (Sigma, St. Louis, Mo.). The cells were loaded with 3 μM Calcium Orange AM ester (Molecular Probes, Eugene, Oreg.) for 90 minutes in the dark. The cells were washed, resuspended in buffer, and incubated for 1 hour to allow de-esterification of the fluorescent probe. The cells were washed twice and placed in a 2 ml quartz cuvet. Calcium release was measured by an LS50B Perkin Elmer Fluorimeter (Ex=549, Em=576) (Perkin Elmer, Wellesley, Mass.) for a 20 minute period. The cells were exposed to the test compounds for 20 minutes and then the fluorescence was recorded. In some experiments, the cells were pretreated with 0.5 μg/ml Pertussis toxin for 5 minutes. FIGS. 7A-7C show Tetrahymena cells loaded with Calcium Orange under fluorescence microscopy (X400). Upon calcium binding to calcium orange, the intensity increases thereby indicating the release of intracellular calcium. FIG. 7A is a control. FIG. 7B shows the calcium release triggered by PsA. FIG. 7C shows Pertussis toxin blocking the calcium release caused by PsA.

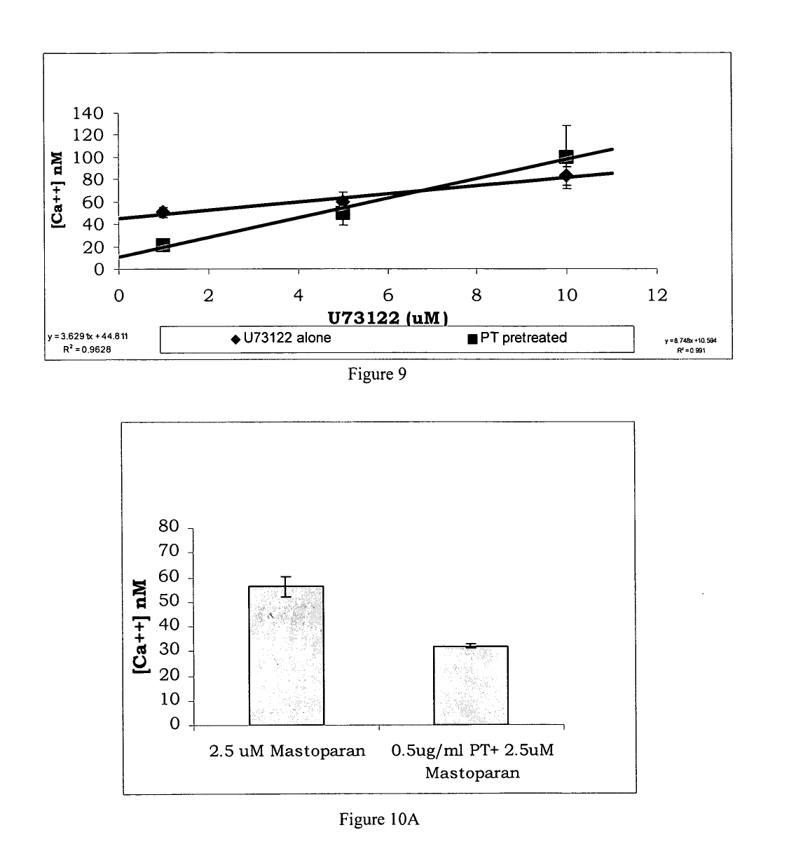

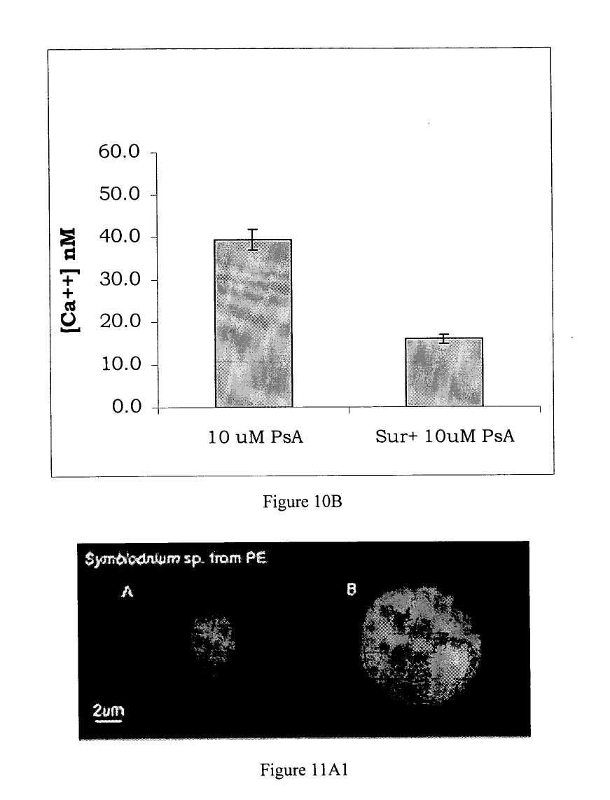

FIG. 8 shows that PsA triggers an intracellular calcium release in a dose dependent manner (ED₅₀=1.9 μM) and Pertussis toxin blocks PsA activity. Cells pretreated with 0.5 μg/ml Pertussis toxin for 5 minutes prior to treatment with PsA inhibited PsA activity. FIG. 9 shows that U73122 causes an intracellular calcium release (ED₅₀=0.6 μM) and that Pertussis toxin does not inhibit U73122 activity. Cells pretreated with 0.5 μg/ml Pertussis toxin for 5 minutes prior to treatment with U73122 did not inhibit U73122 activity. FIG. 10A shows that Pertussis toxin pretreatment (5 minutes) inhibits the effects of mastoparan. Mastoparan does not inhibit PsA. Pertussis toxin and mastoparan both target and modulate Gi/o proteins; Pertussis toxin inhibits and mastoparan activates. FIG. 10B shows that suramin pretreatment (5 minutes) blocks PsA activity. Suramin prevents G protein activation by inhibiting the GDP to GTP exchange.

U73122 shares some of the anti-inflammatory pharmacology of PsA. U73122 is known to inhibit phospholipase C and to cause a selective release of calcium from a subcellular site. As provided herein, the effect of PsA is inhibited or modulated by Pertussis toxin, whereas the effect of U73122 is not. Therefore, PsA acts at the membrane level to activate a Pertussis toxin sensitive receptor that in turn initiates an inhibitory cascade in a manner different from U73122.

During phagosome formation calcium is taken up into the phagosome as part of the formation or taken into a cellular compartment in which it plays a rate limiting role. The mechanism of PsA is to block the calcium uptake so that cytoplasmic calcium rises and such effect is reflected as an increased fluorescence. This effect is also blocked by PT. See FIG. 5; FIGS. 7A, 7B, and 7C; and FIG. 8.

### EXAMPLE 3

### Injury Response Assay—Physical Injury

To determine the protective effect of pseudopterosin compounds against physical injury, low frequency sonic pulses were used to induce injury and release reactive oxygen species as an indicator of injury. The released reactive oxygen species were calibrated to H₂O₂, the least labile of the group. The oxidative burst was measured in Heterocapsa pygamea cells from culture and Symbiodinium spp. cells which were isolated from P. elisabethae live coral by methods known in the art. Specifically, by homogenization in a blender with 0.22 μm filtered seawater and 10 mM EDTA, and filtered through 4 layers of cheesecloth. Algal symbionts were pelleted out by centrifugation at 250×g and subsequently washed 10 times with 40 ml clean filtered seawater and pelleted by centrifugation at 750×g. The cells were further purified on Percoll® (Sigma, St. Louis, Mo.) step gradient of 20%, 40%, and 80% two or more times until less than about 1% impurities were seen using light microscopy. DNA staining using DAPI detected on epifluorescence microscopy was used to detect contaminants due to bacterial or coral cells. Cells isolated from live coral were diluted to a final concentration of about 5×10⁵ cells/ml using a hemocytometer and maintained in filtered seawater.

DNA from purified symbionts was extracted using the NDeasy plant mini prep kit available from (Qiagen, Santa Clarita, Calif.). As described by LaJeunesse (Marine Biology (2002) 141:387-400) denaturing gradient gel electrophoresis (DGGE) was then used to analyze the internal transcribed spacer 2 (ITS 2) sequences to identify the symbiont type occurring in the samples of P. elisabethae. Intracellular Symbiodinium concentrations of pseudopterosin compounds averaged about 0.011 pmol/cell.

Heterocapsa pygmaea is grown in culture in F-1 media without silica. The cells were harvested in log growth phase and diluted to about 5×10⁵ cells/ml using a hemocytometer. For pseudopterosin compound experiments, the cells were incubated with various concentrations of a mixture of PsA, PsB, PsC, and PsD for 1 hour at room temperature. There are no detectable endogenous levels of pseudopterosin compounds in H. pygmacea.

All cells were injured using three 10-second 80W pulses of low frequency sonic sound (20 KHz). H₂O₂ concentration was calculated from a standard curve from DCFH-DA fluorescence (0.05 mM, redox sensitive probe, requires esterase (82 U) for detection of H₂O₂). Fluoresence was measured using a Perkin Elmer LS50B Fluorimeter, excitation 488 nm and emission 525 nm.

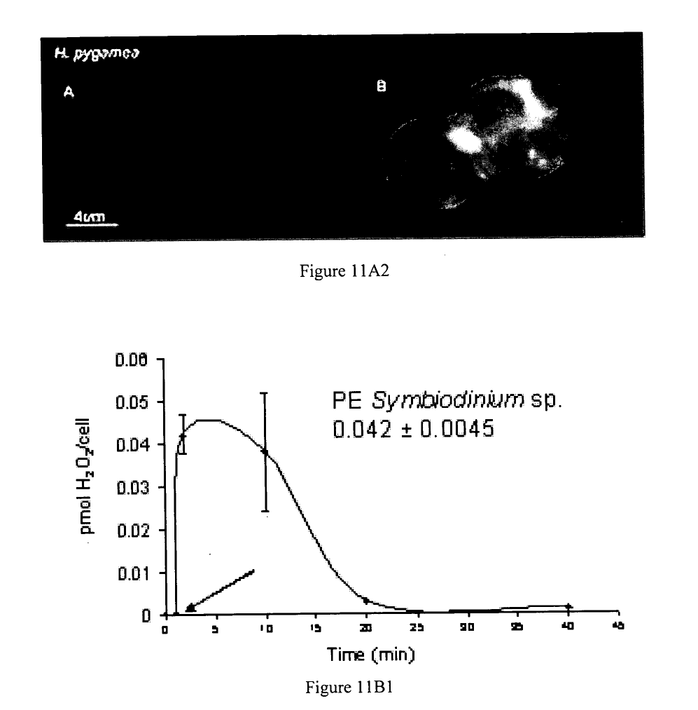

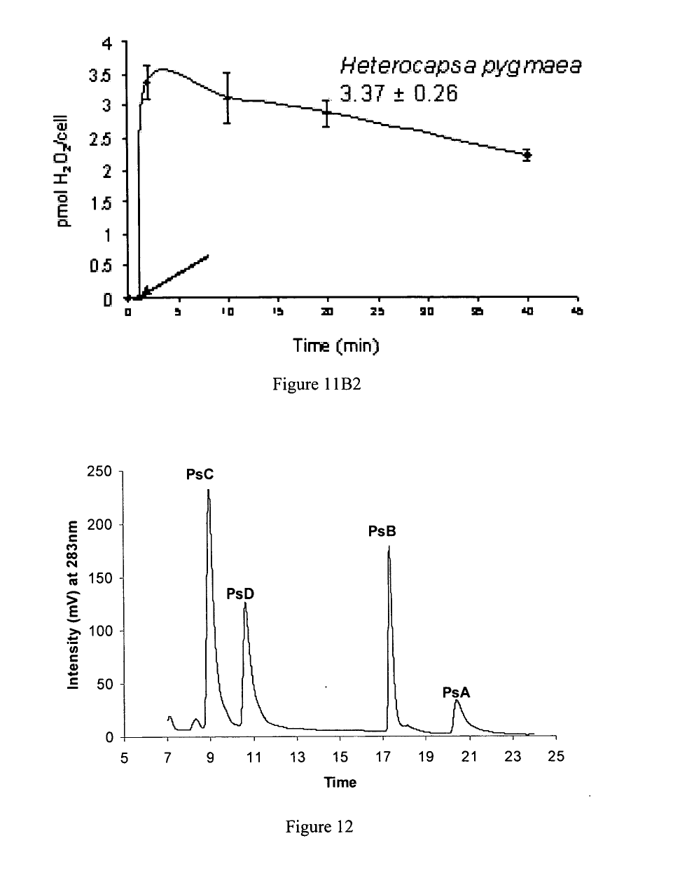

As shown in FIG. 11A1, the concentration of released reactive oxygen species was 0.0082 nmol H₂O₂/min/cell in the injury resistant Symbiodinium and 0.745 mmol H₂O₂/min/cell in the free swimming injury sensitive H. pygmaea, which is greater than about 90 fold.

The lack of sensitivity of the Symbiodinium to ultrasound induced injury may be the result of differences in lipid composition. In particular, about 15% of the lipids in Symbiodinium are comprised of the potent anti-inflammatory diterpenes, pseudopterosin compounds. Thus, the injury response of the H. pygmaea in the presence of various concentrations of a mixture of PsA, PsB, PsC, and PsD was examined.

PsA, PsB, PsC, and PsD are the dominant molecular metabolites found in the injury resistant Symbiodinium. The Ps compounds used in these experiments were prepared from crude extracts using HPLC grade chloroform and ethyl acetate. Crude extracts were partitioned between methanol/water (9:1) and hexanes, followed by partitioning between methanol/water (1:1) and chloroform. The extracts were run on normal phase HPLC with a hexane/ethyl acetate gradient (60:40 to 100% ethyl acetate in 40 minutes) using UV detection at 283 nm. A representative HPLC chromatogram of a Ps mixture is shown in FIG. 12.

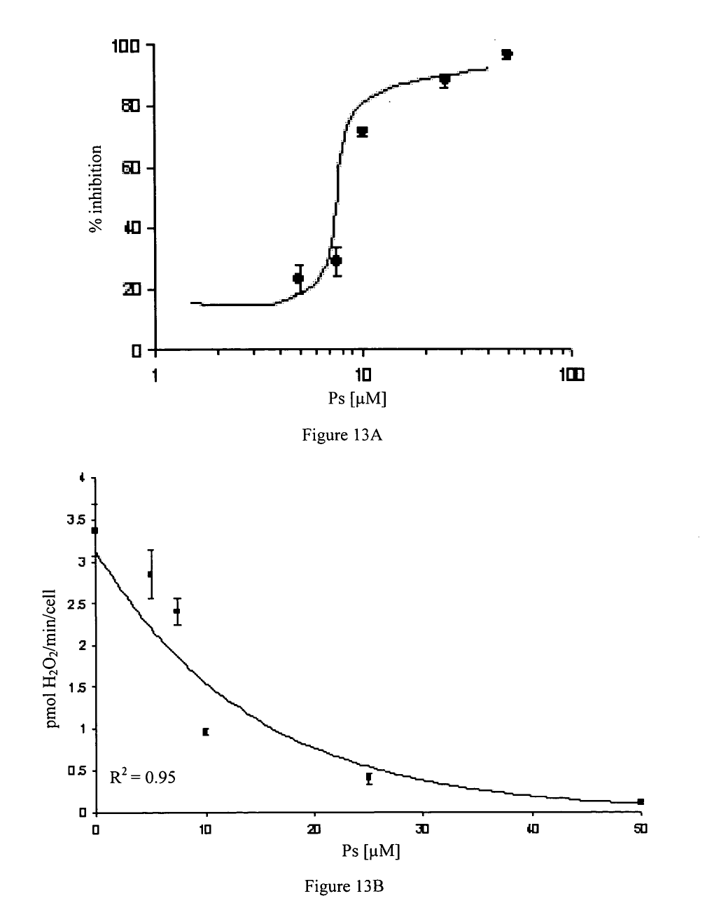

As shown in FIG. 11A2, a 1-hour preincubation with increasing concentrations of the pseudopterosin compound mixture produced an increased resistance to ultrasound injury. Nearly total resistance to injury occurred at concentrations above about 10 μM with an IC₅₀ of about 7.2 μM as provided in FIG. 13.

### EXAMPLE 4

### Injury Response Assay—Chemical Injury

To determine the protective effect of pseudopterosin compounds against chemical injury, mastoparan was administered to test cells and released reactive oxygen species calibrated to H₂O₂ was measured as an indicator of injury.

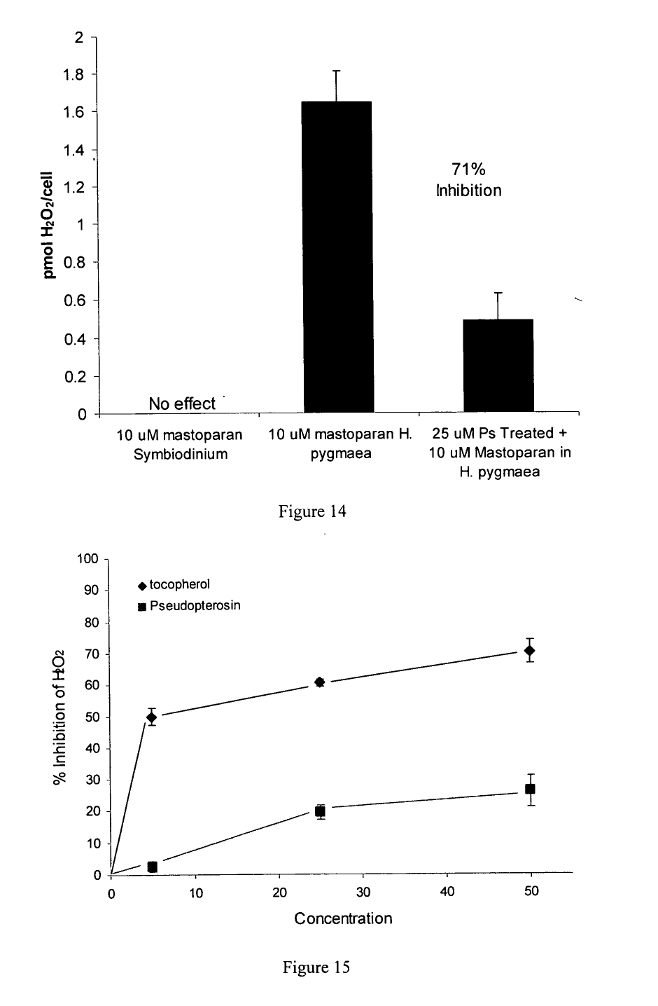

Symbiodinium spp. cells were isolated, prepared and identified as described in Example 3. Heterocapsa pygmaea was grown in culture in F-1 media without silica. The cells were harvested in log growth phase and diluted to about 5×10⁵ cells/ml using a hemocytometer. As shown in FIG. 14, 10 μM of mastoparan had no effect on Symbiodinium spp. cells but did cause a large oxidative burst in H. pygmaea cells. The oxidative burst was prevented or inhibited by about 71% by incubating the H. pygmaea cells with a mixture comprising 25 μM of pseudopterosin compounds for 1 hour prior to exposure to mastoparan.

H₂O₂ concentration was calculated from a standard curve from DCFH-DA fluorescence (0.05 mM, redox sensitive probe, requires esterase (82 U) for detection of H₂O₂). Fluorescence was measured using a Perkin Elmer LS50B Fluorimeter, excitation 488 nm and emission 525 nm.

### EXAMPLE 5

### Injury Response Assay—Radiating UV Injury

To determine the protective effect of pseudopterosin compounds against damaging UV injury, cells preincubated with various doses of pseudopterosin compounds were exposed to 10 minutes of UVC radiation at 254 nm from a UV lamp.

The cells were harvested in log growth phase and diluted to about 5×10⁵ cells/ml using a hemocytometer. ROS concentration was measured in the same manner as previous experiments (DCFH-DA fluorescence (0.05 mM, redox sensitive probe, requires esterase (82 U) for detection of H₂O₂). Fluorescence was measured using a Perkin Elmer LS50B Fluorimeter, excitation 488 nm and emission 525 nm.

Cells exposed to UVC radiation exhibited a release of ROS with a magnitude of 0.461 mmol H₂O₂/cell. The pseudopterosin compounds were able to inhibit the release of ROS with an IC₅₀ of 13 μM.

UVC radiation can disrupt membrane fluidity and cause degradation of microsomal fatty acids and proteins. See Dumont et al. (1992) Free Radical Biology and Medicine 13(3):197-203, which is herein incorporated by reference. The protective effects of pseudopterosin compounds in Heterocapsa pygmaea from UVC radiation further indicates that pseudopterosin compounds exhibit protective and stabilizing features to the membranes and proteins of the cell.

### EXAMPLE 6

### ROS Inhibition

In order to confirm that the effect of pseudopterosin compounds on inhibition of injury response is not due to direct antioxidant effects, the following assay was conducted. A mixture of hydrogen peroxide and water without cells was mixed with the DCFH-DA dye and 82U of esterase as described above. To this mixture the drugs were added and results monitored on the fluorimeter.

Addition of 50 μM pseudopterosin compounds to a cell free mixture of hydrogen peroxide and water did not immediately reduce or scavenge the peroxide or reduce the fluorescent signal of the dye. No significant effect was seen after a 20 minute exposure of the same dose. This effect was compared to a known oxygen radical scavenger, ascorbic acid, which immediately scavenges the hydrogen peroxide radicals in the cell free system. 50 μM ascorbic acid immediately reduced the fluorescent signal by about 60%.

As shown in FIG. 15, the pseudopterosin compounds had no effect on reducing the concentration of hydrogen peroxide in the cell free mixture, indicating that they had no scavenging properties even after a 20 minute incubation. A known scavenger, such as ascorbic acid, reduced the amount of hydrogen peroxide immediately. These results signify that the ROS reducing activity that the pseudopterosin compounds exhibited on the dinoflagellate cells was due to a more complex mechanism of action which may include cell membrane stabilization. Therefore, the reductions in hydrogen peroxide levels by pseudopterosin compounds were not due to direct antioxidant effect.

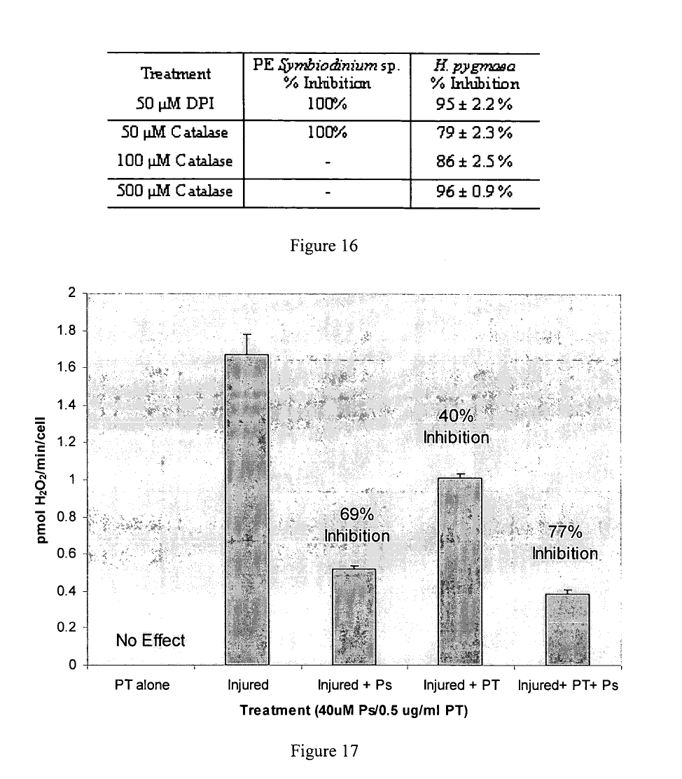

As a control, the injured cells were treated with inhibitors of the ROS pathway. Diphenylene iodonium chloride (DPI) (Sigma, St. Louis, Mo.) inhibited the oxidative burst by about 96% in Heterocapsa pygmaea at 50 μM. DPI is a nonreversible inhibitor of NADPH oxidase. As shown in FIG. 16, the results indicate that the burst response in these dinoflagellates has similar elements to the higher plant oxidative stress pathway.

### EXAMPLE 7

### Pertussis Toxin Effects on Physical Injury in Heterocapsa Pygmaea

To test the effects of Pertussis Toxin (PT) on prevention or stimulation of the oxidative burst due to physical injury in H. pygmaea, 0.5 μg/ml PT was incubated with 3×10⁶ cells/ml for 1 hour prior to injury by ultrasonic sound. The use of PT was to investigate the involvement of G-proteins in the oxidative burst. PT catalyzes the ADP-ribosylation of the alpha subunits of the heterotrimeric guanine nucleotide regulatory proteins Gi, Go, and Gt. This prevents the G protein heterotrimers from interacting with receptors, thereby blocking their coupling and activation.

On its own, PT did not cause an oxidative burst, thereby indicating that the ROS pathway is not sensitive to this toxic effect. As illustrated in FIG. 17, PT did moderately inhibit the oxidative burst due to physical injury when the cells were pretreated prior to injury. This inhibition was not as strong as the effects of the pseudopterosin compounds when the cells were incubated and injured under the same conditions.

To test whether the inhibitory effects of a pseudopterosin mixture (Ps, a mixture of Pseudopterosin compounds A-D made as previously described) were G-protein based, cells were incubated with PT and the pseudopterosin mixture for 1 hour prior to injury. PT did not inhibit the effects of the pseudopterosin mixture, but rather decreased the oxidative burst by about an additional 8%. The G-protein inhibitory pathway was not turned on by PT in this model.

### EXAMPLE 8

### Pseudopterosin Compound Effects on Physical Injury in Tetrahymena Thermophila

The effect of a pseudopterosin mixture (a mixture of Pseudopterosin compounds A-D made as previously described) was measured in Tetrahymena thermophila cells exposed to non-lethal, non-lysing ultrasonic sound. This experiment was performed in order to investigate if the pseudopterosin mixture (Ps) could protect Tetrahymena cells against physical injury.

Tetrahymena cells were washed in 10 mM HEPES, 50 μM CaCl₂ buffer (pH 7.4) and density was adjusted to 500,000 cells/ml. The cells were then incubated for one hour with different concentrations of the pseudopterosin mixture. After incubation, the cells were exposed to non-lethal, non-lysing ultrasonic sound for 7 seconds (40%). Injury was measured as a release of H₂O₂ (oxidative burst) released from Tetrahymena cells by fluorescent spectroscopy.

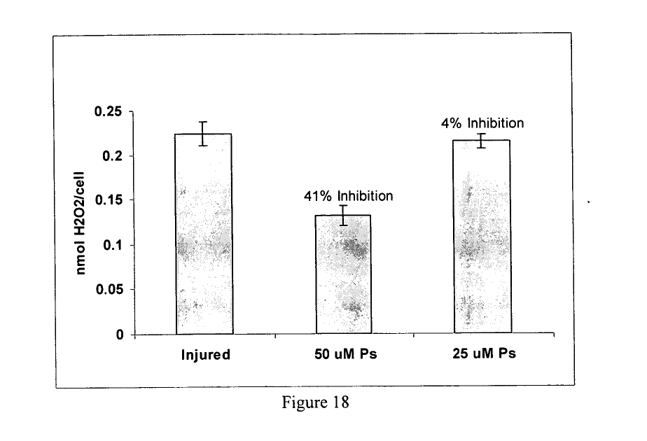

As shown in FIG. 18, injury was inhibited by about 41% when Tetrahymena cells were treated with 50 μM of the pseudopterosin mixture and only about 4% when treated with 25 μM of the pseudopterosin mixture.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations and modifications may be made within the scope of the present invention. Accordingly, the present invention is not limited to the specific embodiments as illustrated herein, but is only limited by the following claims.

1. A method for preventing, inhibiting, decreasing, or modulating phagocytosis in a cell which comprises administering to the cell an effective amount of at least one pseudopterosin compound.

2. The method of claim 1, wherein the cell is a Tetrahymena spp. cell or a Heterocapsa spp. cell.

3. The method of claim 1, wherein the pseudopterosin compound is Pseudopterosin A (PsA), Pseudopterosin B (PsB), Pseudopterosin C (PsC), Pseudopterosin D (PsD), Pseudopterosin E (PsE), Pseudopterosin F (PsF), Pseudopterosin G (PsG), Pseudopterosin H (PsH), Pseudopterosin I (PsI), Pseudopterosin J (PsJ), Pseudopterosin K (PsK), Pseudopterosin L (PsL), Pseudopterosin M (PsM), Pseudopterosin N (PsN), Seco-Pseudopterosin A (SPsA), Seco-Pseudopterosin B (SPsB), Seco-Pseudopterosin C(SPsC), Seco-Pseudopterosin D (SPsD), Seco-Pseudopterosin E (SPsE), or Elisabethatriene.

4. The method of claim 1, wherein the pseudopterosin compound is Pseudopterosin A (PsA), Pseudopterosin B (PsB), Pseudopterosin C (PsC), or Pseudopterosin D (PsD).

5. The method of claim 1, wherein the pseudopterosin compound is Pseudopterosin A (PsA).

6. The method of claim 1, wherein the effective amount ranges from about 0.1 μM to about 100 μM.

7. The method of claim 6, wherein the effective amount ranges from about 1 μM to about 50 μM.

8. The method of claim 7, wherein the effective amount ranges from about 2 μM to about 25 μM.

9. The method of claim 8, wherein the effective amount ranges from about 2.5 μM to about 10 μM.

10. The method of claim 1, and further comprising administering a calcium ionophore, an inhibitor of PLC activation, or both.

11. A method of treating, preventing, or inhibiting a disease or disorder associated with phagocytosis in a subject which comprises administering to the subject a therapeutically effective amount of at least one pseudopterosin compound.

12. A method for inducing, increasing, or modulating calcium mobilization in a cell which comprises administering to the cell an effective amount of at least one pseudopterosin compound.

13. The method of claim 12, wherein the cell is a Tetrahymena spp. cell or a Heterocapsa spp. cell.

14. The method of claim 12, wherein the pseudopterosin compound is Pseudopterosin A (PsA), Pseudopterosin B (PsB), Pseudopterosin C (PsC), Pseudopterosin D (PsD), Pseudopterosin E (PsE), Pseudopterosin F (PsF), Pseudopterosin G (PsG), Pseudopterosin H (PsH), Pseudopterosin I (PsI), Pseudopterosin J (PsJ), Pseudopterosin K (PsK), Pseudopterosin L (PsL), Pseudopterosin M (PsM), Pseudopterosin N (PsN), Seco-Pseudopterosin A (SPsA), Seco-Pseudopterosin B (SPsB), Seco-Pseudopterosin C(SPsC), Seco-Pseudopterosin D (SPsD), Seco-Pseudopterosin E (SPsE), or Elisabethatriene.

15. The method of claim 12, wherein the pseudopterosin compound is Pseudopterosin A (PsA), Pseudopterosin B (PsB), Pseudopterosin C (PsC), or Pseudopterosin D (PsD).

16. The method of claim 12, wherein the pseudopterosin compound is Pseudopterosin A (PsA).

17. The method of claim 12, wherein the effective amount ranges from about 0.1 μM to about 100 μM.

18. The method of claim 17, wherein the effective amount ranges from about 1 μM to about 50 μM.

19. The method of claim 18, wherein the effective amount ranges from about 1 μM to about 25 μM.

20. The method of claim 19, wherein the effective amount ranges from about 1 μM to about 10 μM.

21. The method of claim 12, and further comprising administering an inhibitor of PLC activation.

22. A method of treating, preventing, or inhibiting a disease or disorder associated with calcium mobilization in a subject which comprises administering to the subject a therapeutically effective amount of at least one pseudopterosin compound.

23. A method of treating, preventing, or inhibiting an injury to a cell or a tissue which comprises administering to the subject a therapeutically effective amount of at least one pseudopterosin compound to the cell or the tissue.

24. The method of claim 23, wherein the injury is a physical injury, a chemical injury, a radiation injury, or a combination thereof.

25. The method of claim 23, wherein the pseudopterosin compound is Pseudopterosin A (PsA), Pseudopterosin B (PsB), Pseudopterosin C (PsC), Pseudopterosin D (PsD), Pseudopterosin E (PsE), Pseudopterosin F (PsF), Pseudopterosin G (PsG), Pseudopterosin H (PsH), Pseudopterosin I (PsI), Pseudopterosin J (PsJ), Pseudopterosin K (PsK), Pseudopterosin L (PsL), Pseudopterosin M (PsM), Pseudopterosin N (PsN), Seco-Pseudopterosin A (SPsA), Seco-Pseudopterosin B (SPsB), Seco-Pseudopterosin C(SPsC), Seco-Pseudopterosin D (SPsD), Seco-Pseudopterosin E (SPsE), or Elisabethatriene.

26. The method of claim 23, wherein the pseudopterosin compound is Pseudopterosin A (PsA), Pseudopterosin B (PsB), Pseudopterosin C (PsC), or Pseudopterosin D (PsD).

27. The method of claim 23, wherein the pseudopterosin compound is Pseudopterosin A (PsA).

28. The method of claim 23, wherein the therapeutically effective amount ranges from about 0.1 μM to about 100 μM.

29. The method of claim 28, wherein the therapeutically effective amount ranges from about 1 μM to about 50 μM.

30. The method of claim 29, wherein the therapeutically effective amount ranges from about 2.5 μM to about 25 μM.

31. The method of claim 30, wherein the therapeutically effective amount ranges from about 5 μM to about 15 μM.

32. The method of claim 23, and further comprising administering at least one supplementary active compound.
